(19) United States
(12) Patent Application Publication
(10) Pub. No.: US 2014/0326245 A1
(43) Pub. Date: Nov. 6, 2014

(54) Medical Face Mask with Sealing Strip

(71) Applicant: Mei-Sheng Teng, Kowloon (HK)

(72) Inventor: Mei-Sheng Teng, Kowloon (HK)

(21) Appl. No.: 14/243,195

(22) Filed: Apr. 2, 2014

Related U.S. Application Data
(63) Continuation-in-part of application No. 13/933,821, filed on Jul. 2, 2013.
(60) Provisional application No. 61/819,969, filed on May 6, 2013.

(51) Int. Cl. A61M 16/06

(52) U.S. Cl. CPC A61M 16/0605
USPC 128/206.13

(57) ABSTRACT

The present invention relates to a face mask for filtering contaminants from the air that is suitable for medical applications. The face mask includes an air-permeable body having an inner surface, wherein the body is sized to cover the nose and mouth of a subject when worn by the subject, an inner seal connected to said inner surface of said body, wherein said inner seal is sized to form a perimeter around a wearer's nostrils and mouth, at least one strap connected to said body for securing said face mask to said subject's face, and a deflector layer connected to the inner surface of said body, wherein the deflector redirects at least a portion of the exhaled air down into the lower regions of the air-permeable body.

### CROSS-REFERENCE TO RELATED APPLICATIONS

The present application claims priority under 35 U.S.C. 119(e) to U.S. Provisional Patent Application No. 61/819,969 filed May 6, 2013, which is incorporated by reference herein in its entirety. The present application is also a continuation-in-part of U.S. application Ser. No. 13/933,821 filed Jul. 2, 2013, which claims priority under 35 U.S.C. 119(e) to U.S. Provisional Patent Application No. 61/819,969 filed May 6, 2013, the entire disclosures of which are each incorporated by reference herein in their entirety.

### BACKGROUND OF THE INVENTION

Face masks are often used as personal protective equipment in a variety of situations, such as during medical treatment or in dusty environments. Medical personnel, such as nurses and surgeons, often need to wear face masks when providing care to a patient. Such face masks are generally designed to filter airborne contaminants from the air being inhaled by the user in order to protect the user from inhaling pathogens and other contaminants, while also protecting people near the user from inhaling contaminants exhaled by the user. Such airborne contaminants may include aerosolized saliva, bacteria, viruses, dust, or any other type of particle that can be suspended in air.

Face masks currently available are typically produced with one of two types of protection. Both types of masks are made by a variety of different manufacturers. The type of mask with the highest level of protection is the N95 mask, which refers to an efficiency rating determined by the National Institute for Occupational Safety and Health (NIOSH). The “N95” designation corresponds to a mask that blocks about 95% of particles that are 0.3 microns or larger. With testing for proper fit, these masks can provide good protection against inhaling harmful particles.

On the other hand, the type of mask used most frequently in healthcare environments is the “surgical” or “procedure” mask. The surgical masks presently available do not provide a complete seal with the user's face, resulting in a lower filtering capability compared to N95 masks. However, these surgical masks are significantly less expensive than N95 masks. These surgical masks are typically constructed from a flat, pleated filter material that spreads when the mask is worn. When the mask is secured to the user's face, gaps between the filter material and the wearer's face can occur. While these masks may protect the wearer from particles impacting directly against the mask, they offer little or no protection from contaminants suspended in air that can pass around the edges of the mask into the wearer's lungs.

Further, it is well recognized that one of the biggest problems for wearers of current surgical masks is the unwanted flow of moist, exhaled breath up the inner surface of the face of the mask, exiting towards their eyes. This exhaled breath can irritate the wearer's eyes, and if the wearer is also wearing glasses, significant fogging can occur. While anti-fogging strips are available for placement onto typical surgical masks, they unfortunately reduce the surface area for air to exit the mask, driving even more exhaled air out towards the eyes.

Thus, there is a continuing need in the art for a surgical face mask that provides an improved seal with the user's face, and prevents or significantly reduces exhaled breath from escaping out the top region of the mask toward the wearer's eyes. The present invention addresses this unmet need in the art.

### SUMMARY OF THE INVENTION

The present invention relates to a face mask for filtering contaminants from air. The mask includes an air-permeable body having an inner surface, wherein the body is sized to cover the nose and mouth of a subject when worn by the subject, an inner seal connected to the inner surface of the body, wherein the inner seal is sized to form a perimeter around a wearer's nostrils and mouth, at least one strap connected to the body for securing the face mask to the subject's face, and a deflector layer connected to the inner surface of the body, wherein the deflector redirects at least a portion of the exhaled air down into the lower regions of the air-permeable body. In one embodiment, the inner seal is configured to form a seal around the subject's nose and mouth when worn by the subject. In another embodiment, the inner seal includes at least one leaflet for conforming a portion of the inner seal to the subject's nose. In another embodiment, the inner seal comprises silicone. In another embodiment, the at least one strap is connected to two or more extensions protruding from the side of the face mask. In another embodiment, the air-permeable body is slightly rigid. In another embodiment, an exterior surface of the air-permeable body includes at least one ridge to provide the slight rigidity. In another embodiment, the deflector layer is air-impermeable. In another embodiment, the deflector is at least partially coated with silicone along the inner surface of the face mask body.

The present invention also relates to a face mask that includes an air permeable body having an inner surface, wherein the body is sized to cover the nose and mouth of a subject when worn by the subject, an inner seal connected to the inner surface of the body, wherein the inner seal is sized to form a perimeter around a wearer's nostrils and mouth, at least one strap connected to the body for securing the face mask to the subject's face, and an air impermeable layer formed along the top portion of the inner surface of the body, wherein the air impermeable layer redirects at least a portion of the exhaled air down into the lower regions of the air-permeable body. In one embodiment, the inner seal is configured to form a seal around the subject's nose and mouth when worn by the subject. In another embodiment, the inner seal comprises at least one leaflet for conforming a portion of the inner seal to the subject's nose. In another embodiment, the inner seal comprises silicone. In another embodiment, the at least one strap is connected to two or more extensions protruding from the side of the face mask. In another embodiment, the body is slightly rigid.

The present invention also relates to a face mask that includes an air-permeable body having an inner surface, wherein the body is sized to cover the nose and mouth of a subject when worn by the subject, an inner seal connected to the inner surface of the body, wherein the inner seal is sized to conform to the sides of the wearer's nose and cheek regions under the wearer's eyes, at least one strap connected to the body for securing the face mask to the subject's face, and a deflector layer connected to the inner surface of the body, wherein the deflector redirects at least a portion of the exhaled air down into the lower regions of the air-permeable body. In one embodiment, the inner seal comprises at least one leaflet for conforming a portion of the inner seal to the subject's nose. In one embodiment, the inner seal comprises silicone. In one embodiment, the deflector layer is an air-impermeable layer. In one embodiment, the deflector layer comprises a silicone coating.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an illustration showing the outside of a facial mask, according to one embodiment of the present invention.

FIG. 2 is an illustration showing a perspective view of the inside of a facial mask, according to one embodiment of the present invention.

FIG. 3 is an illustration showing the inside of a facial mask, according to one embodiment of the present invention.

FIG. 4 is an illustration showing the inside of a facial mask, according to one embodiment of the present invention.

FIG. 5 is an illustration showing the outside of a facial mask, according to one embodiment of the present invention.

FIG. 6 is an illustration showing a perspective view of the outside of a facial mask, according to one embodiment of the present invention.

FIG. 7 is an illustration showing a perspective view of the inside of a facial mask, according to one embodiment of the present invention.

FIG. 8 is an illustration showing a perspective view of the inside of a facial mask, according to one embodiment of the present invention.

FIG. 9 is a set of illustrations showing the inner seal of a facial mask, according to one embodiment of the present invention, wherein the inner seal is positioned on a user's face.

FIG. 10 is an illustration showing a perspective view of the inside of a facial mask, according to one embodiment of the present invention.

FIG. 11 is a set of illustrations showing the inside of various embodiments of the facial mask of the present invention.

FIG. 12 is a set of illustrations of facial masks. FIG. 12A shows an inside view and side view of a facial mask, according to one embodiment of the present invention. FIG. 12B shows an inside view and side view of a common facial mask, i.e., a mask currently available in the art.

FIG. 13 is an illustration showing a perspective view of the inside of a facial mask, according to one embodiment of the present invention.

FIG. 14 is an illustration showing a perspective view of the outside of a facial mask, according to one embodiment of the present invention.

FIG. 15 is a set of views of an exemplary mask having a deflector therein. FIG. 15A is a perspective view of the inner surface of the exemplary mask,

FIG. 15B is a side view of the exemplary mask, and 15C is a perspective view of the outer surface of the exemplary mask.

FIG. 16 is a set of views of an exemplary mask having an air-impermeable layer therein. FIG. 16A is a perspective view of the inner surface of the exemplary mask, and 16B is a perspective view of the outer surface of the exemplary mask.

FIG. 17 is a set of perspective views (17A and 17B) of an exemplary mask having a partial inner seal, a deflector and an air-impermeable layer therein.

### DETAILED DESCRIPTION

It is to be understood that the figures and descriptions of the present invention have been simplified to illustrate elements that are relevant for a clear understanding of the present invention, while eliminating, for the purpose of clarity, many other elements found in medical face masks. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods, materials and components similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “contaminant,” “particle,” and the like may be used interchangeably herein, and refer to a constituent or impurity in the air or environment surrounding the user of the mask of the present invention that can adversely affect the user if inhaled.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The present invention relates to a face mask that filters contaminants from air during inhalation and exhalation. The face mask of the present invention provides a more complete seal than face masks in the prior art, thereby decreasing the risk of contaminants bypassing the face mask's filter during inhalation or exhalation. In one embodiment, the face mask comprises a seal within the mask that fits around the nose and mouth of the user, i.e., an inner seal. In another embodiment, the face mask comprises two seals for sealing the mask to the user's face: an inner seal and also a seal around the edge of the mask, i.e., an outer seal. The mask also comprises an air-permeable body that serves to filter contaminants from air and a mechanism for securing the mask to the user's face, for example a strap that connects to the mask and can be positioned around the user's head.

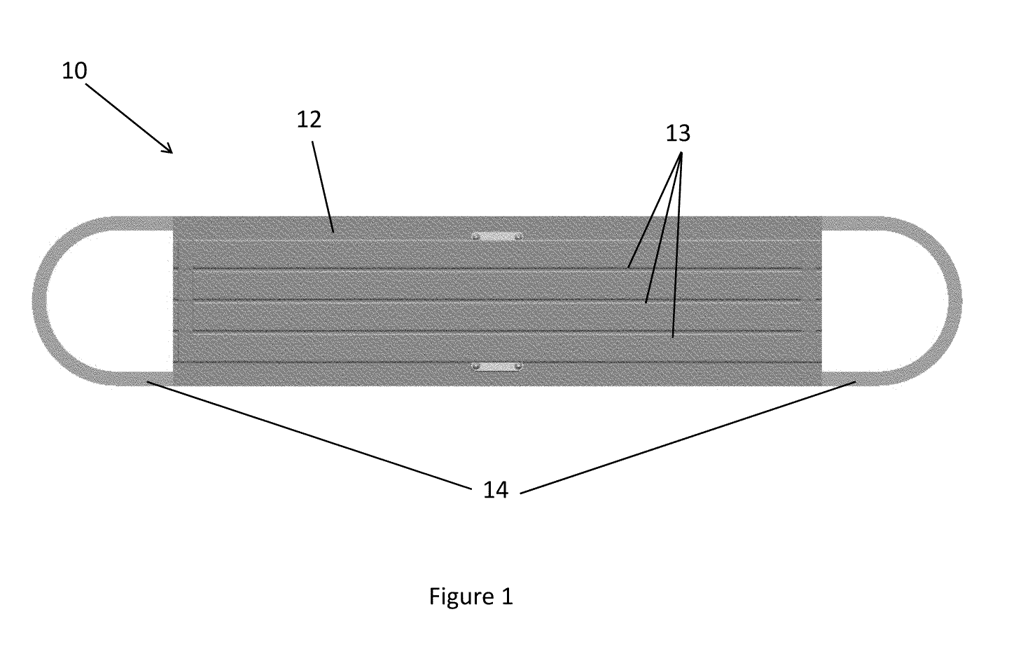

Referring now to the drawings, FIG. 1 is an illustration of one embodiment of a face mask 10 of the present invention. FIG. 1 shows the outside of face mask 10, comprising an air-permeable body 12 and straps 14. Straps 14 may be used to secure face mask 10 to the user's face by placing straps 14 around the user's ears. Air-permeable body 12 of face mask 10 comprises a number of pleats 13 that allow for air-permeable body 12 to be stretched and configured without damaging or compromising the structural integrity of air-permeable body 12.

In one embodiment of the face mask of the present invention, the air-permeable body 12 comprises a non-woven material or fabric. Non-woven materials are materials comprising fibers or filaments that are bound together mechanically, thermally, or chemically. The fibers or filaments are typically composed of synthetic polymers, but can be composed of any material suitable for both filtering particulates and contacting human skin, as would be understood by a person with ordinary skill in the art.

In one embodiment, straps 14 are configured to be placed around the ears of the user. In another embodiment, the straps are configured to be placed around the head or neck of the user. In one embodiment, the straps comprise an elastomeric material suitable for securing the mask to the user's face. In another embodiment, the straps further comprise a clasping mechanism for securing the straps around the user's head or neck.

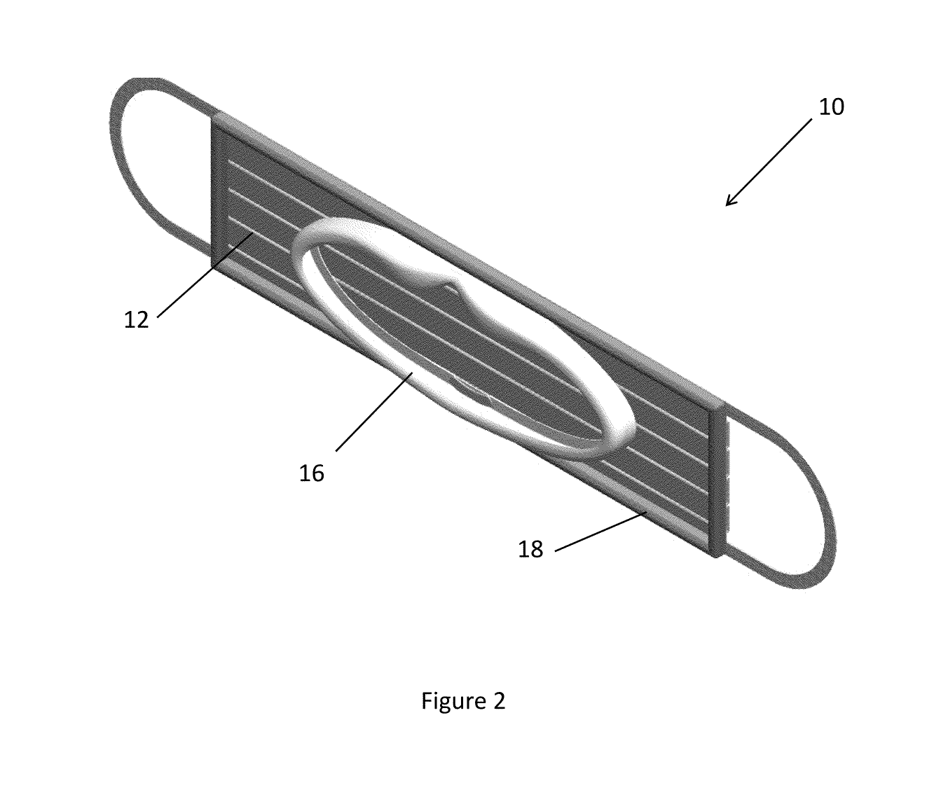

Referring to FIG. 2, an illustration of a perspective view of the inside of face mask 10 is shown, i.e., the side of face mask 10 that is in contact with the user's face when face mask 10 is worn. Face mask 10 comprises air-permeable body 12, an inner seal 16 and an outer seal 18.

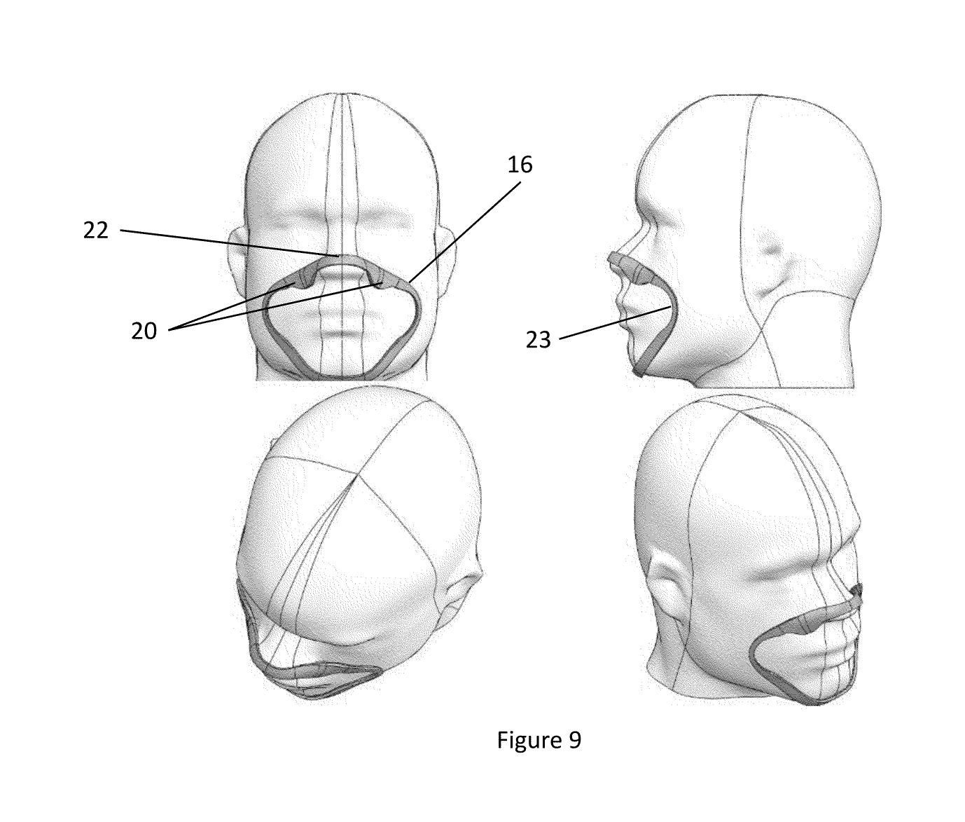

In one embodiment of the present invention, the inner seal 16 of the face mask of the present invention comprises a relatively soft material that forms a substantially circular or elliptical shape around the user's nose and mouth when the mask is worn. Referring to FIG. 9, a series of illustrations shows inner seal 16 conforming to a user's face. In one embodiment, inner seal 16 further comprises wider or thicker regions, also referred to herein as leaflets 20, that conform to the shape of the user's face on either side of the user's nose to improve the sealing characteristics of inner seal 16. In one embodiment, inner seal 16 comprises a region 22 between leaflets 20 that forms a seal with the bridge of the user's nose. In various embodiments, the length of region 22 may be sized appropriately so that leaflets 20 form a seal with the sides of the user's nose, i.e., so an optimal seal is formed in the area of the user's face where there are creases between the user's nose and cheeks. Further, in one embodiment, inner seal 16 comprises a side loop 23 that fits around the side of user's cheek in order to provide an optimal seal with the user's face. In one embodiment, the inner seal is made of silicone. In another embodiment, the inner seal may comprise any elastomeric material such as a thermoplastic elastomer (TPE), or a combination of materials such as TPE with ethylene-vinyl acetate (EVA) and/or polyurethane (PU), suitable for forming an air-tight seal around the user's nose and mouth.

In one embodiment, the outer seal 18 of the present invention comprises a relatively soft material that can conform to the contours of the user's face in order to form an air-tight seal between the air-permeable body of the present invention and the user's face. In one embodiment, the outer seal comprises foam made from a TPE/EVA blended resin. In another embodiment, the foam of the outer seal comprises polyurethane. In another embodiment, the outer seal may comprise any material, or combination of materials, suitable for forming an air-tight seal between the mask and the user's face. In yet another embodiment, the face mask of the present invention may comprise only one seal, wherein the single seal is an outer seal instead of an inner seal.

In one embodiment, a mild adhesive may be used in conjunction with the inner seal, outer seal, or both, in order to further improve the quality of the seal between the mask and the user's face. In another embodiment, the inner seal, outer seal, or both, may comprise an elastomeric material that is formulated to produce a sticky or tacky effect, in order to further improve the quality of the seal between the mask and the user's face

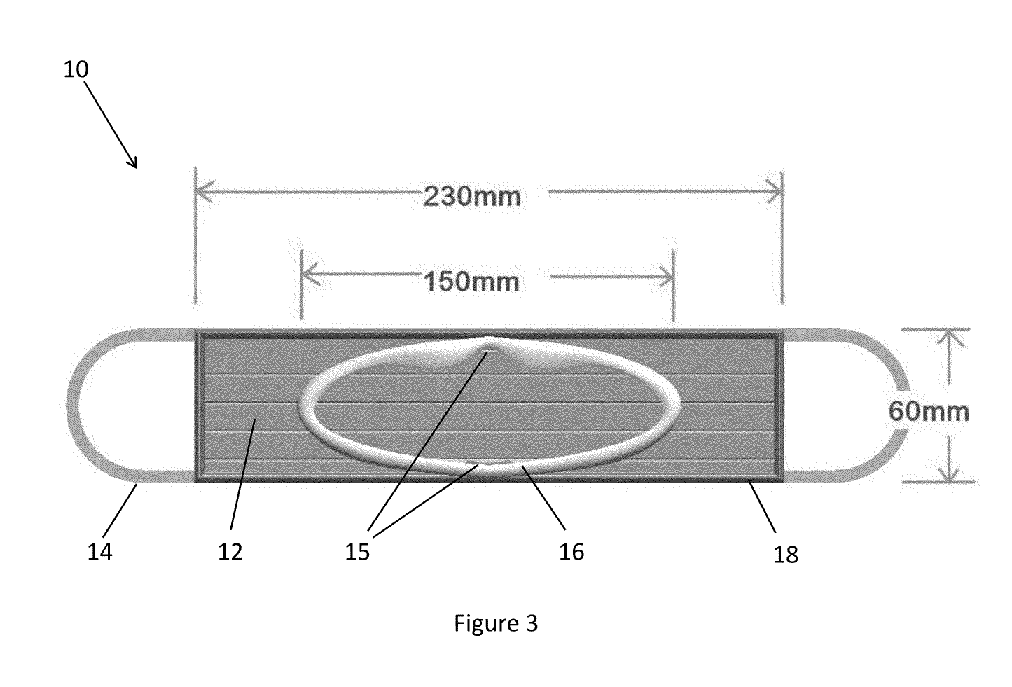

Referring to FIG. 3, an illustration of the inside of face mask 10 is shown. Face mask 10 comprises straps 14, inner seal 16, and outer seal 18 Inner seal 16 is attached to face mask 10 at two points via connector strips 15. The dimensions of various parts of an exemplary embodiment of face mask 10 are also shown in FIG. 3, in millimeters (mm). FIG. 3 shows one possible configuration of face mask 10, in which air-permeable body 12 and inner seal 16 are not stretched vertically, i.e., the inner seal is in a relaxed state. Specifically, the height of air-permeable body 12 is about 60 mm at all points, while the width of inner seal 16 is about 150 mm. The overall width of air-permeable body 12 is about 230 mm.

In various embodiments, the face mask of the present invention can be configured or adjusted to fit the contours of the user's face. In one embodiment, pleats 13 in the air-permeable body 12 allow for the body to be stretched to match the dimensions of the user's face. In one embodiment, outer seal 18 is suitably flexible to stretch or adjust in conjunction with air-permeable body 12 without separating or detaching from air-permeable body 12.

In one embodiment, inner seal 16 is attached to air-permeable body 12 via at least one connector strip 15. In a preferred embodiment, inner seal 16 is attached to air-permeable body 12 via two connector strips, such that the inner seal 16 can maintain sufficient flexibility to change shape when air-permeable body 12 is adjusted vertically, while remaining connected to air-permeable body 12. In one embodiment, each connector strip 15 serves to fasten inner seal 16 to air-permeable body 12 in a single location, thereby leaving the remaining portion of inner seal 16 unconnected to air-permeable body 12. In such an embodiment, most of inner seal 16 is free-floating, i.e., unconnected to air-permeable body 12 or any other part of face mask 10, in order to allow inner seal 16 to adjust and conform to the user's face when worn by the user. In one embodiment, inner seal 16 is forced against air-permeable body 12 when worn by the user, thereby forming a seal between inner seal 16 and air-permeable body 12, in addition to the seal formed between inner seal 16 and the user's face. In such an embodiment, the seal formed between inner seal 16 and air-permeable body 12 is a mechanical seal resulting from the pressure of inner seal 16 against air-permeable body 12 caused by the force of the user's face pushing against inner seal 16.

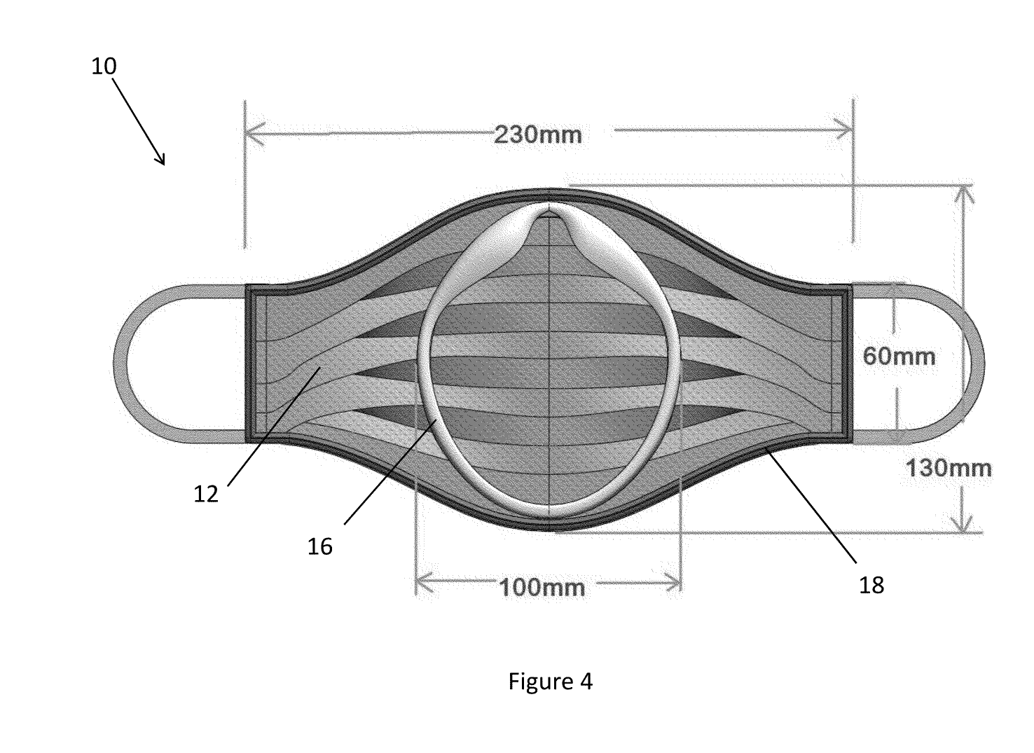

Referring to FIG. 4, an illustration of the inside of face mask 10 is shown, in which a portion of face mask 10 has been stretched to match the dimensions of a user's face, i.e., to cover the user's nose and mouth. Specifically, the height of air-permeable body 12 is about 130 mm at its fully expanded point, while the width of inner seal 16 is about 100 mm (compared to 150 mm in FIG. 3). Notably, the overall width of air-permeable body 12 is about 230 mm, i.e., the same as in FIG. 3. Therefore, as depicted in FIG. 4, inner seal 16 and a portion of air-permeable body 12 have been stretched vertically without substantially changing the overall width of air-permeable body 12. Further, the shape of outer seal 18 has been adjusted accordingly with air-permeable body 12.

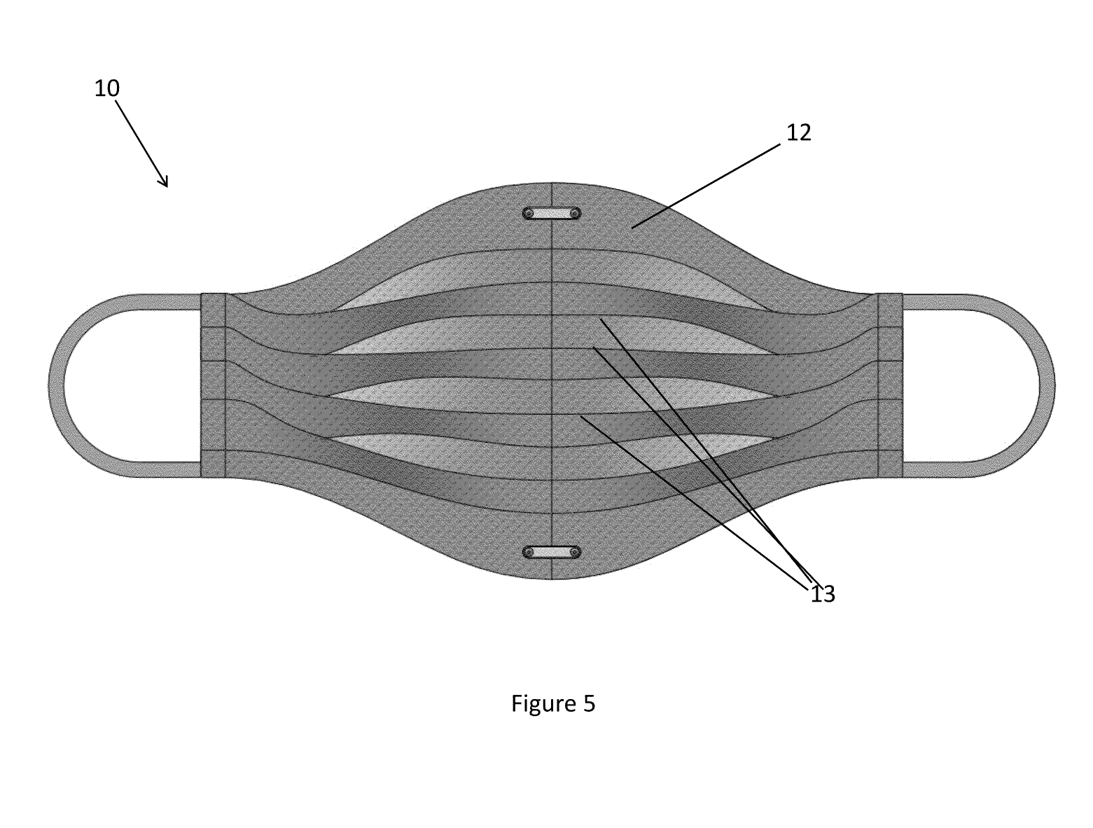

Referring to FIG. 5, an illustration of the outside of face mask 10 is shown, in which air-permeable body 12 has been configured to cover the user's nose and mouth. As described herein, air-permeable body 12 comprises pleats 13 that enable air-permeable body 12 to be stretched without reducing or compromising the structural integrity of air-permeable body 12.

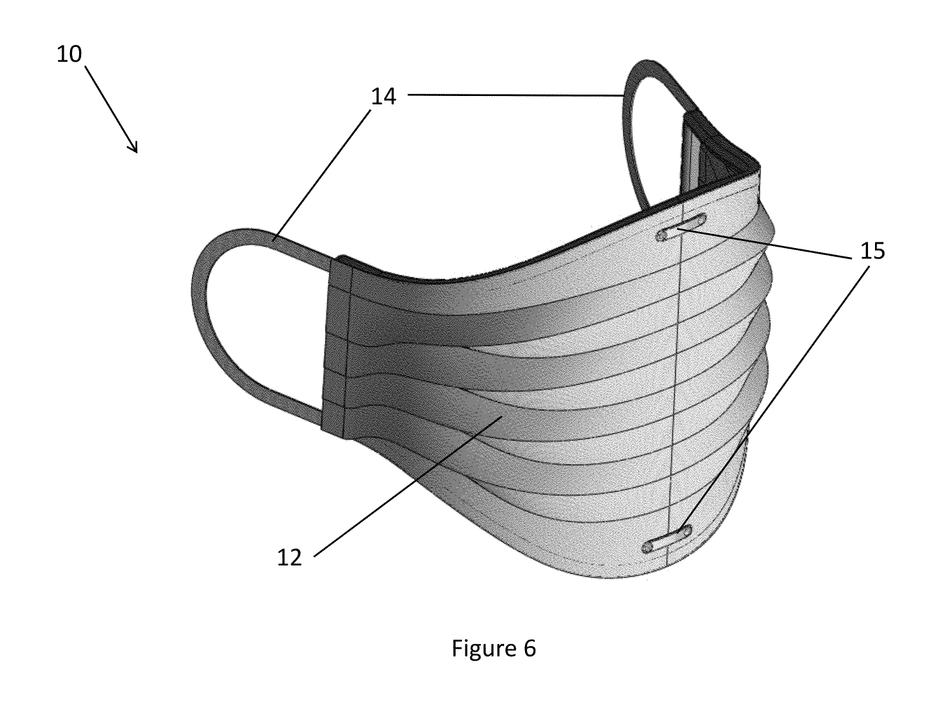

Referring to FIG. 6, an illustration of a perspective view of the outside of face mask 10 is shown. Connector strips 15 are used to attach the inner seal 16 (not shown) to air-permeable body 12 of face mask 10. In this view, face mask 10 is configured to the approximate shape of face mask 10 when it is secured to a user's face. In this embodiment, straps 14 may be secured around the user's ears to secure face mask 10 to the user's face, while air-permeable body 12 is configured to cover the user's nose and mouth.

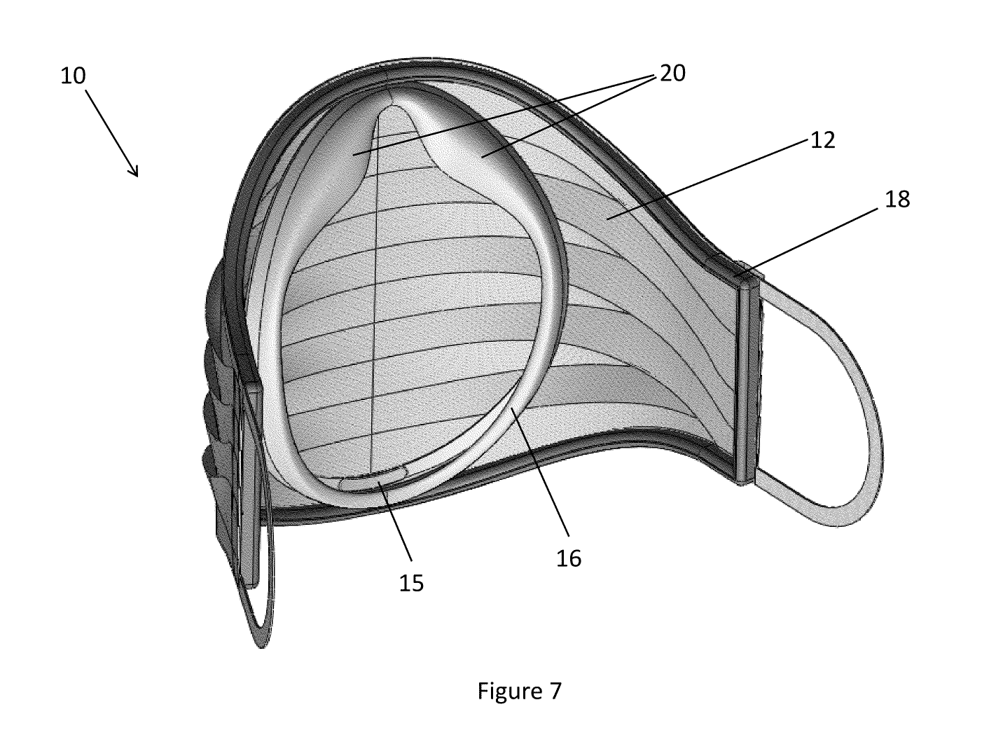

Referring to FIG. 7, an illustration of a perspective view of the inside of face mask 10 is shown. In this embodiment, air-permeable body 12 and inner seal 16 are configured to cover the user's nose and mouth, similarly to FIG. 6. Outer seal 18 maintains the shape of the perimeter of air-permeable body 12. Connector strips 15 connect inner seal 16 to air-permeable body 12. Further, inner seal 16 comprises leaflets 20, i.e., a wider region of the inner seal, that provide an optimal fit of inner seal 16 to the bridge of the user's nose. As contemplated herein, inner seal may include additional leaflet regions to provide additional depth and/or thickness to better conform to the user's facial contours, such as around the cheeks, mouth and portion of the chin.

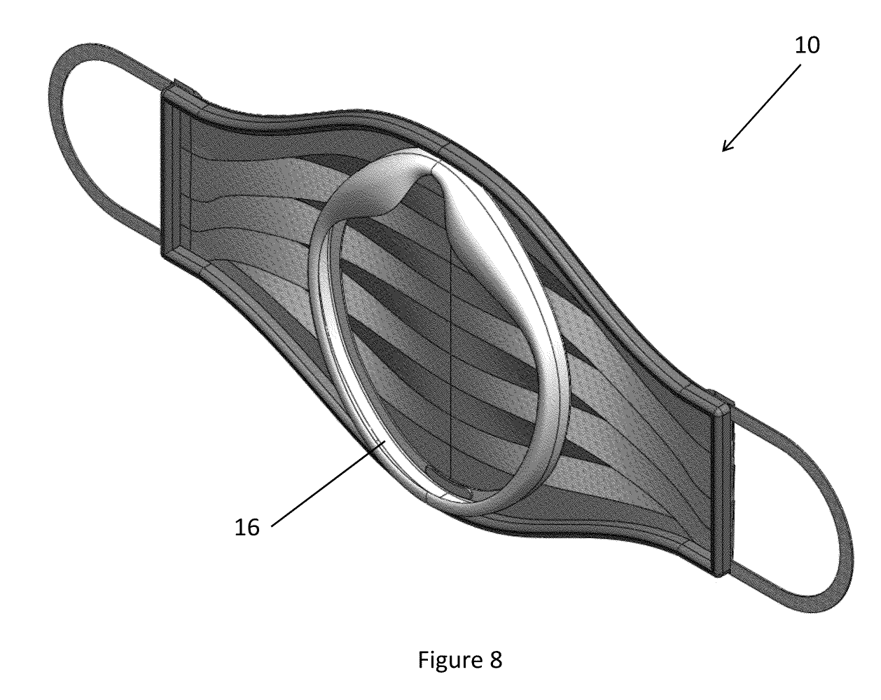

Referring to FIG. 8, an illustration of a perspective view of face mask 10, comprising inner seal 16 is shown.

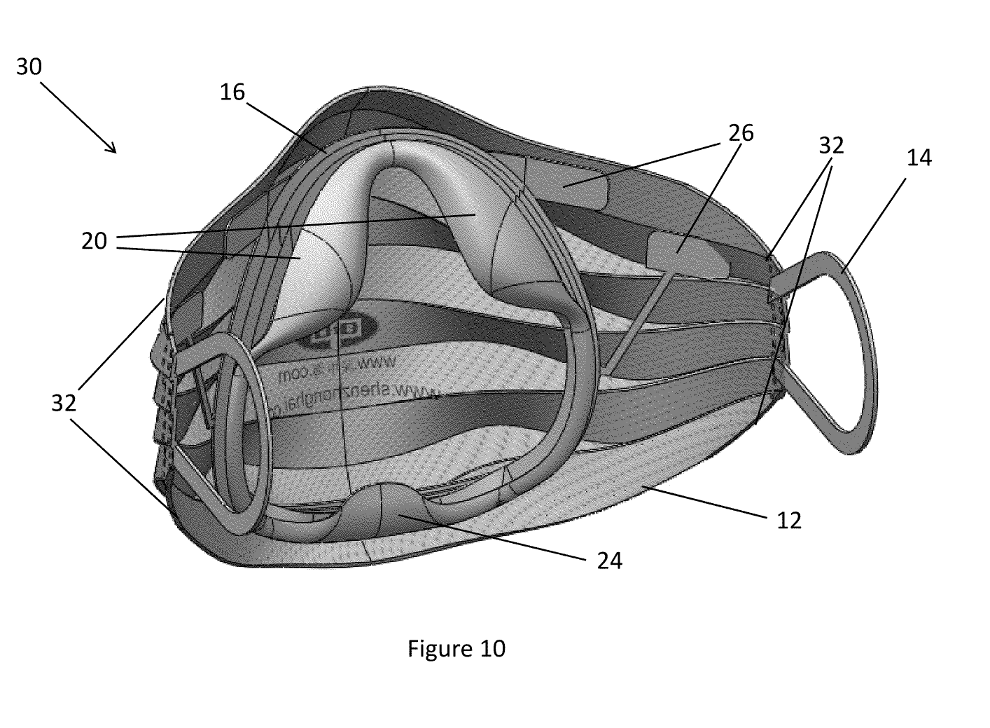

Referring to FIG. 10, an illustration of the inside of another embodiment of the face mask of the present invention is shown. Face mask 30 comprises an air-permeable body 12, straps 14, and an inner seal 16 Inner seal 16 is connected to air-permeable body 12 via connector tabs 26. Inner seal 16 comprises a bottom region 24 that is wider than other areas of inner seal 16, much like leaflets 20. Bottom region 24 may be suitable for forming a seal with an area on user's chin. Air-permeable body 12 of face mask 30 comprises angled regions 32, wherein the height of air-permeable body 12 reduces from its widest point to its narrowest point near the part of face mask 30 where straps 14 are connected. In one embodiment, angled region 32 is created by manufacturing air-permeable body 12 in a shape that is generally rectangular, but with a sloping angle near the ends of the rectangle, i.e., a reduced width at each of the four corners of the rectangle as compared to the width at the center of the rectangle.

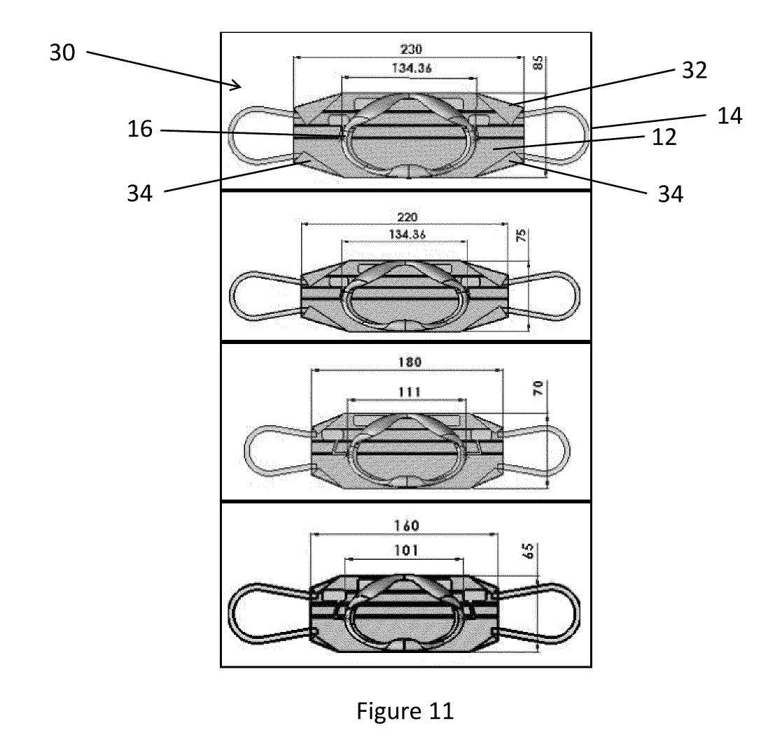

In another embodiment, angled regions 32 are created by manufacturing air-permeable body 12 in the shape of a rectangle, then folding each of the four corners of the rectangle toward the inside surface of air-permeable body 12. An example of such an embodiment is shown in FIG. 11. In FIG. 11, four embodiments of face mask 30 are shown, wherein each embodiment comprises the same shape and features, but with different dimensions, as indicated. Angled regions 32 are shown on air-permeable body 12, wherein a portion of air-permeable body 12 has been folded to create flaps 34 at each corner of air-permeable body 12. Accordingly, a region near each of the four corners of air-permeable body 12 has two layers of material where flaps 34 have been folded. Flaps 34 may improve the seal between the user's face and air-permeable body 12 by increasing the stiffness of at least a portion of air-permeable body 12, which may prevent undesired folding of face mask 30 when worn by the user. Further, flaps 34 may be used to secure, or aid in securing, straps 14 to air-permeable body 12. In one embodiment, flaps 34 may be secured to the inner surface of air-permeable body by any means that would be understood by a person of ordinary skill, for example, but not limited to, an adhesive or a clamp.

In addition, the width of the region of air-permeable body 12 that has been folded to form flaps 34 is less than other regions of air-permeable body 12. In one embodiment, this reduced width at the ends of air-permeable 12 may improve the seal of the face mask of the present invention to the user's face by providing a narrower region where straps 14 can connect to face mask 30. Such a narrow region may reduce the possibility of folds or gaps being created when the straps are attached to the user, for example when straps 14 are placed around the user's ears.

As shown in FIG. 11, in various embodiments, face mask 30 may comprise different lengths, for example 160 mm, 180 mm, 220 mm, or 230 mm. Further, face mask 30 may comprise different widths at its widest point, for example 65 mm, 70 mm, 75 mm, or 85 mm. Further still, inner seal 16 may comprise different widths, for example 101 mm, 111 mm, or 134.36 mm. However, as would be understood by a person with ordinary skill in the art, the present invention is not limited to the dimensions described herein.

In one embodiment, face mask 30 differs from face mask 10 by eliminating the outer seal of face mask 10. Accordingly, flaps 34 may create an air-tight seal between the user's face and face mask 30 without the need for an outer seal. However, in another embodiment, at least a portion of the perimeter of air-permeable body 12 of face mask 30 comprises an outer seal.

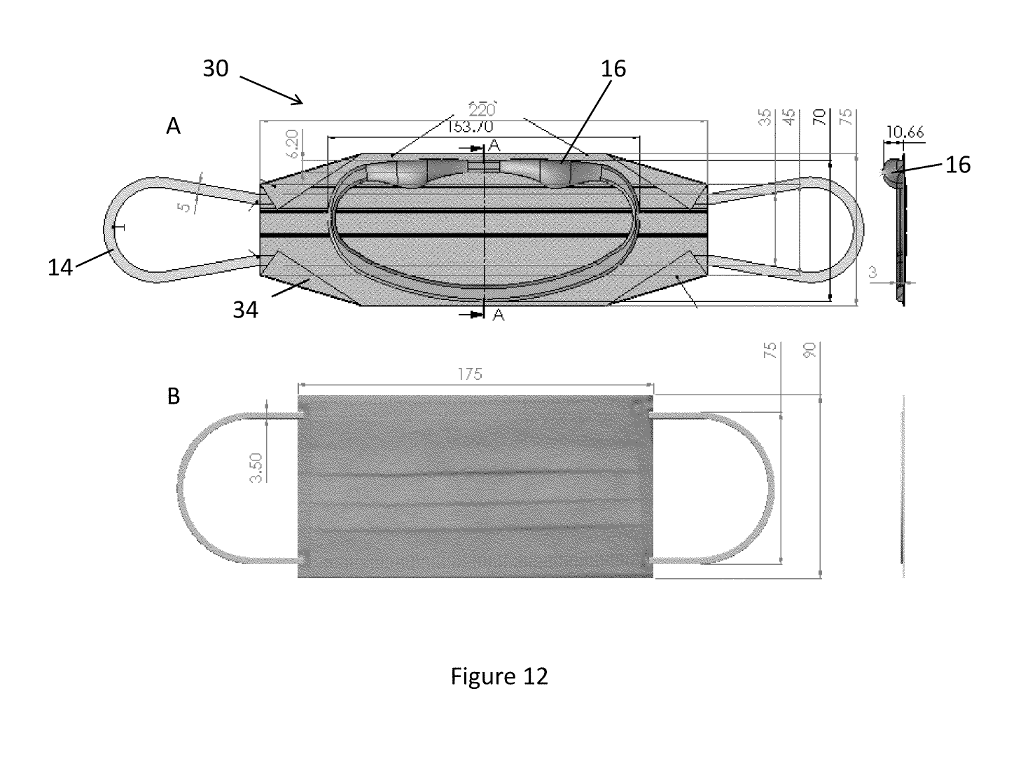

Referring to FIG. 12, a comparison between an embodiment of the face mask of the present invention and an embodiment of a face mask currently available is shown, including some relevant dimensions of both face masks. The diagram at the left of FIG. 12A shows the inside of face mask 30 of the present invention, wherein the diagram at the right shows the profile of face mask 30. The image at the left of FIG. 12 B shows the inside of a face mask of a type currently available wherein the image at the right shows the profile of the face mask currently available. As can been seen in FIG. 12, this embodiment of face mask 30 differs from the currently available face mask in a number of respects such as, but not limited to: face mask 30 is substantially longer than the face mask in FIG. 12B (220 mm vs, 175 mm, excluding the size of the ear straps); face mask 30 comprises an inner seal 16, whereas the mask in FIG. 12B does not; and ear straps 14 of face mask 30 are connected to the mask to allow for flaps 34 to be folded, while the mask in FIG. 12B does not have such flaps, and could not have such flaps, because the ear straps of the mask in FIG. 12B are connected close to the edge of the corners of the rectangle.

Accordingly, these differences between face mask 30 and the face mask in FIG. 12B, at least, result in improved sealing of face mask 30 to the user's face when compared to the mask in FIG. 12B. For example, the longer size of face mask 30, along with flaps 34, optimizes the seal of the mask at the sides of the user's face, i.e., the area of the user's face closest to the user's ears. In addition, the inner seal of face mask 30 creates an air-tight seal around the user's nose and mouth. Conversely, the face mask in FIG. 12B allows for gaps between the mask and the user's face when worn by the user, created, for example, by the body of the mask folding or bunching, or by the lack of a mechanism properly adhere the mask to the different contours of the user's face. Accordingly, the mask in FIG. 12B cannot provide an optimal, air-tight seal to all portions of the user's face.

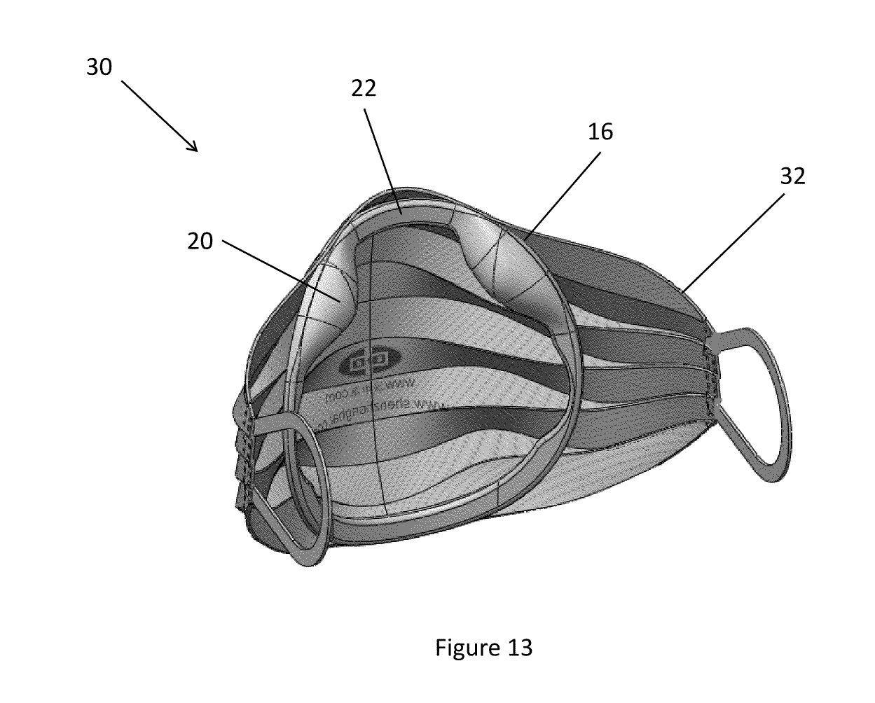

Referring to FIG. 13, an illustration of one embodiment of the inside of face mask 30 is shown, comprising angled regions 32. In this embodiment, an embodiment of inner seal 16 is shown, wherein inner seal 16 comprises a region 22 between leaflets 20 that is wide enough to form an optimal seal with the bridge of the user's nose.

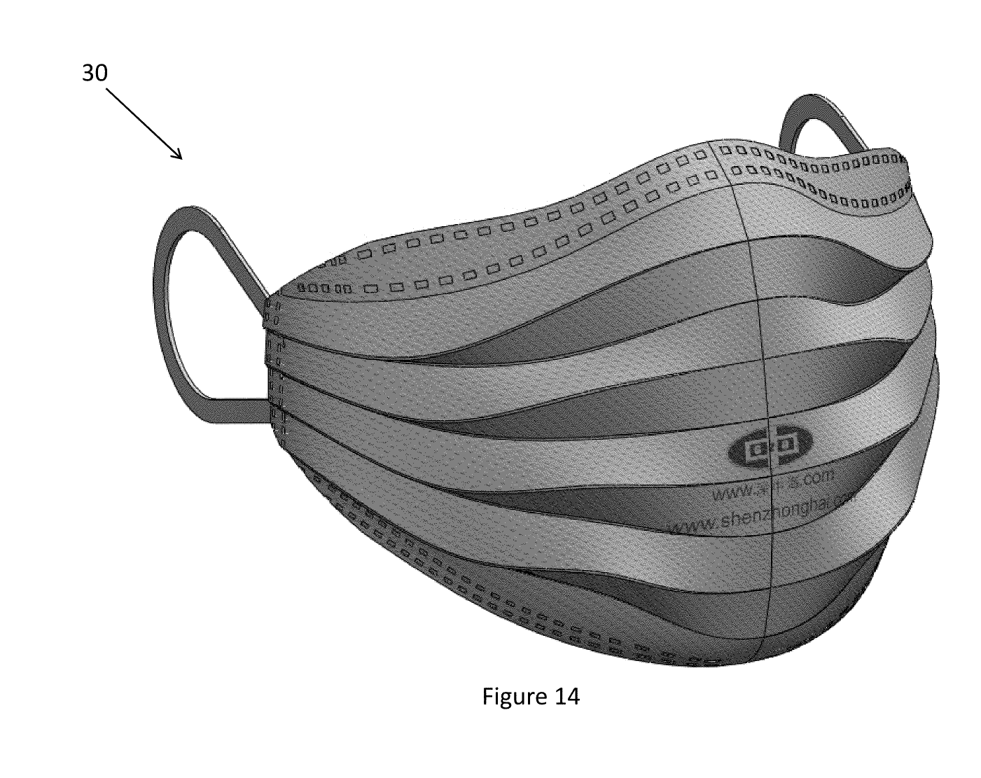

Referring to FIG. 14, an illustration of a perspective view of the outside of face mask 30 is shown.

In another embodiment of the present invention, the mask is uniquely structured to prevent or significantly reduce exhaled breath from escaping out the top region of the mask toward the wearer's eyes. It is well recognized that one of the biggest problems for wearers of surgical masks is the unwanted flow of moist, exhaled breath up the inner surface of the face of the mask, exiting towards their eyes. This exhaled breath can irritate the wearer's eyes, and if the wearer is also wearing glasses, significant fogging can occur. While anti-fogging strips are available for placement onto typical surgical masks, they unfortunately reduce the surface area for air to exit the mask, driving even more exhaled air out towards the eyes.

Air flow within a surgical mask is determined by several factors. For example, air flow is affected by how well the mask seals at all contact points along its edge. Further, air flow is effected by the resistance of the filter material, which itself is determined by both how tight the filter material is (meaning the size of the openings in the material) and the surface area across which the air flow is dispersed. For a surgical mask to be effective in preventing bacteria, viruses or other particulates from passing through the mask material in both directions, the openings in the material need to be very small. As a consequence of this, the smaller sized openings that filter out the unwanted particulates inevitably increase resistance to exhaled breath passing through and exiting the face of the mask. This results in a temporary increase in air pressure between the wearer's face and the mask, and directs the air flow to any path of lesser resistance. For existing masks, the path of least resistance is at the top portion of the mask around the wearer's eyes and bridge of the nose.

Accordingly, the mask of the present invention prevents or reduces air flow toward the eyes of the wearer by creating a superior seal around the bridge of the nose and upper cheeks under the eyes. Further, exhaled breath is redirected away from the upper portion of the mask, thereby allowing air to flow back toward the face of the mask and exit the mask preferably through the filter material.

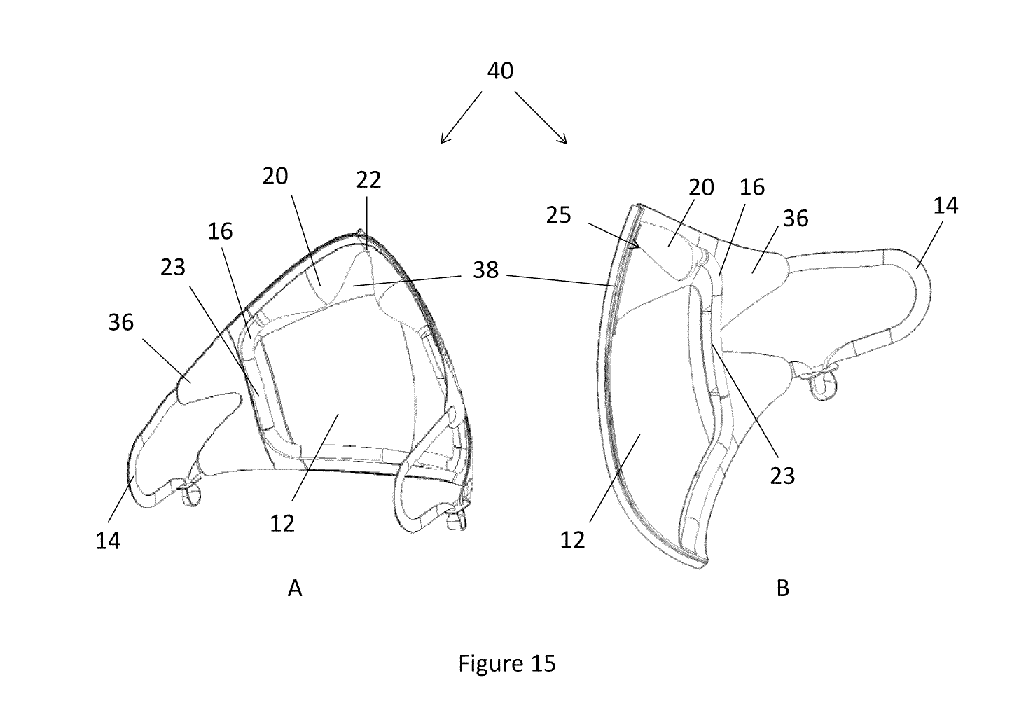

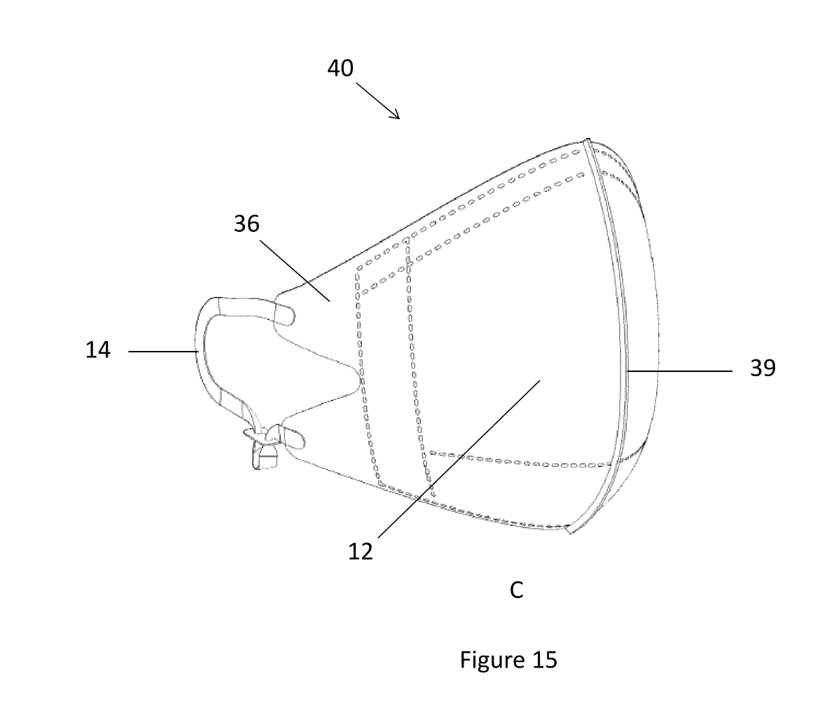

For example, as shown in FIGS. 15A and 15B, mask 40 may include an inner seal 16, shaped and constructed in a similar manner as described and shown in the embodiments of FIGS. 9 and 10 herein. Face mask 40 similarly comprises an air-permeable body 12, straps 14, along with inner seal 16, which forms a superior seal around all contact points of inner seal 16 to the wearer's face Inner seal 16 of mask 40 is constructed from a relatively soft material that forms a substantially circular or elliptical shape around the user's nose and mouth when the mask is worn, such that it sufficiently conforms to the contours of all contact points to the wearer's face. To enhance the strength of the seal and to promote additional comfort, inner seal 16 comprises leaflets 20, which are wider and/or thicker regions that better conform to the shape of the wearer's face on either side of the wearer's nose Inner seal 16 also comprises bridge region 22 between leaflets 20 that may vary in length to account to the particular structure and size of the wearer's nose. Further, inner seal 16 comprises side loop 23 that fits around the side of the wearer's cheek in order to provide an optimal seal with the wearer's face.

Air-permeable body 12 may be foldable, as shown in other various embodiments described herein, or it may be moderately rigid, such that body 12 may substantially hold its generally cupped shape when little or no force is put against it. To provide a moderate amount of rigidity, the outside surface of body 12 may include a ridge 39 formed from the mating of two portions of body 12 in construction of mask 40 or from the pinching and fixing of a ridge-like line vertically and centrally along the outer surface of body 12, such that body 12 can substantially hold its generally cupped shape, as shown in FIG. 15C. Likewise, body 12 may flex slightly when worn by a user, such that a superior seal is formed against the wearer's face, yet body 12 still maintains its generally cupped shape to provide a distance between the mouth and nostrils of the wearer's face and allow air to flow between the wearer's face and the inner surface of body 12. To improve a smooth and flush contact of inner seal 16 against the wearer's face, the sides of mask 40 may include extensions 36 to which strap 14 is affixed. Extensions 36 prevent or reduce any bunching of material of mask 40 when worn, such that inner seal 16 is not offset at an undesirable starting angle when contacting the wearer's face.

Mask 40 may further include a deflector sheet 38, positioned along the top portion of the inner surface of body 12 and optionally in contact with the bottom portion 25 of leaflets 20 and bridge region 22 of inner seal 16. In one embodiment, deflector sheet 38 contacts enough of bottom surface 25 of leaflets 20 and bridge region 22 to create a ridge or a small pocket that effectively blocks air flow from pushing out the top of inner seal 16 toward the eyes, and instead re-directs the air flow back down into the open space near the face of the mask, where the air can exit through the material of body 12. Alternatively, deflector sheet 38 may include a folded region, such that the line of the fold is at the top of mask 40, and leaflets 20 and bridge 22 of inner seal 16 are attached to the flap created beyond the fold line. Thus, the flap and fold region may create a pocket that deflects air traveling up the inner surface of body 12 back down into the open space near the face of the mask, where the air can suitably exit the mask through the material of body 12.

Accordingly, deflector sheet 38 is attached near the top edge of mask 40 so that when the wearer exhales against the resistance caused by the material of body 12, the air flow is pushed up along the front surface of body 12 towards the eyes. As it passes between the material of body 12 and deflector sheet 38 and reaches the top edge, the air cannot escape in that direction and is therefore directed back down towards the front of the nose and mouth. This brief period is sufficient time for the pressure inside mask 40 between the mask and the wearer's face to drop as the other exhaled breath passes through and exits the material of body 12 without causing the moist air to fog the glasses or irritate the eyes of the wearer. Deflector 38 may extend downward along the inner surface of body 12, such that the deflector covers about the top 1-50 mm of the inner surface of body 12. In other embodiments, the deflector covers about the top 1-40 mm, the top 1-30 mm, the top 1-20 mm, the top 1-15 mm, the top 1-10 mm, or the top 1-5 mm.

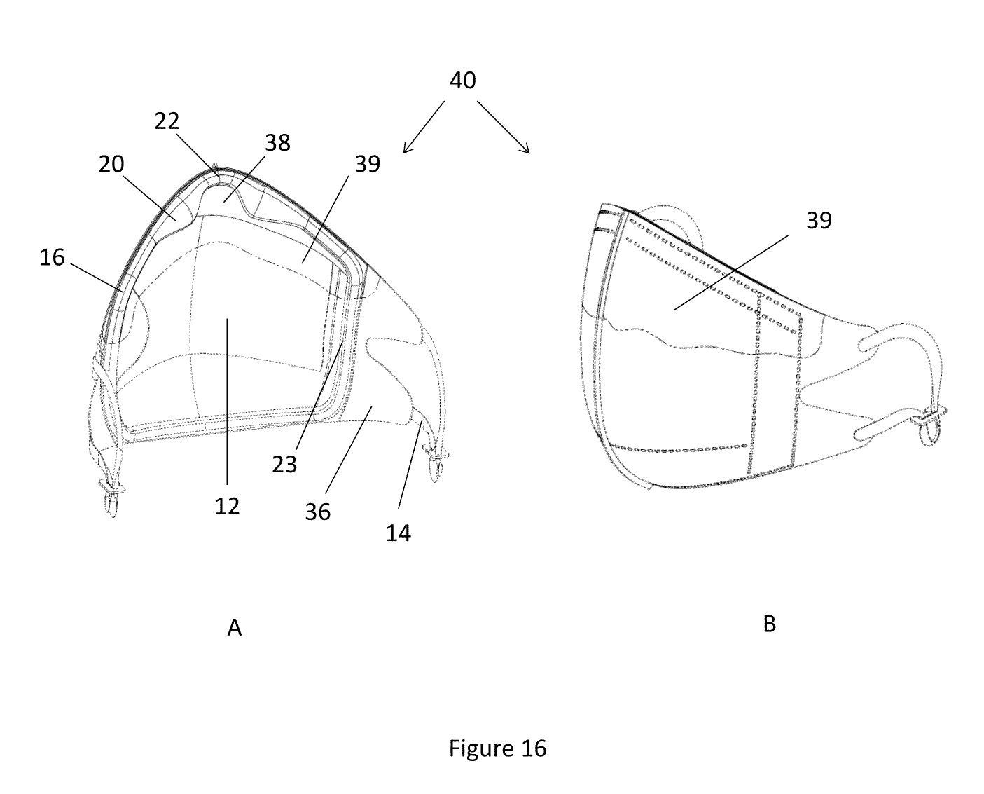

In another embodiment, deflector 38 may be coated with an air-impermeable material, such as silicon. Accordingly, as shown in FIG. 16A, an impermeable layer 39 is formed by the silicon coating, such that no air can pass through either impermeable layer 39 or through nose bridge 22 and leaflets 20 of inner seal 16. This results in the exhaled air being forced back down to escape either through the lower portions of the mask filter material 12 or alternatively through the sides or bottom of the mask. In some embodiments, impermeable layer 39 only covers a portion of deflector 38. In other embodiments, impermeable layer 39 covers substantially all of deflector 38. In still other embodiments, impermeable layer 39 covers all of deflector 38 as well as at least a portion of body 12 going down towards the middle region of mask 40. In yet another embodiment, impermeable layer 39 may be on the outside surface of mask 40, as shown in FIG. 16B. In other embodiments, impermeable layer 39 is on both the inner surface and outer surface of mask 40. In some embodiments the impermeable layer may be formed as part of the inner seal, and may be molded at the same time as the inner seal is molded and positioned onto the mask body.

It should also be appreciated that impermeable layer 39 may be applied to mask 40 without an additional deflector 38 component. For example, a silicon layer may be applied to the top portion of the mask, such that it extends downward below nose bridge region 22 and leaflets 20 of inner seal 16. Because exhaled air cannot escape through either impermeable layer 39 or through nose bridge region 22 and leaflets 20 of inner seal 16, the exhaled air is forced back down into the lower region of the mask, where it can escape through the filter material body 12 without causing the wearer's glasses to fog or to irritate the wearer's eyes. Air impermeable layer 39 may extend downward along the inner surface of body 12, such that the impermeable layer covers about the top 1-50 mm of the inner surface of body 12. In other embodiments, the impermeable layer covers about the top 1-40 mm, the top 1-30 mm, the top 1-20 mm, the top 1-15 mm, the top 1-10 mm, or the top 1-5 mm.

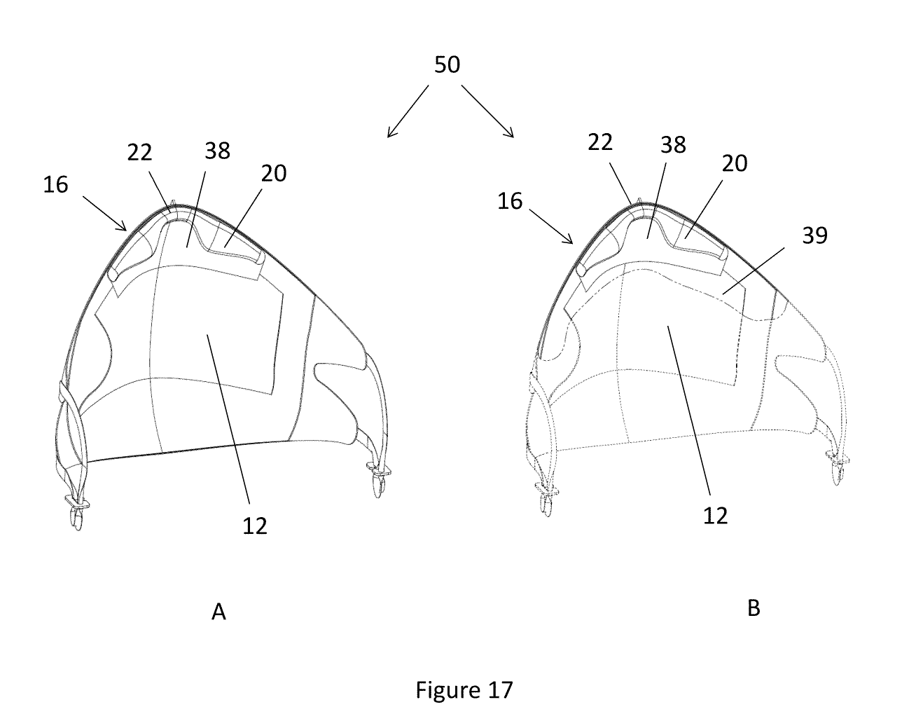

In another embodiment, as shown in FIGS. 17A and 17B, mask 50 may include a partial inner seal 16 that includes nose bridge region 22 and leaflets 20. Accordingly, because the sides and bottom portion of inner seal 16 are absent, exhaled air can escape from the sides and bottom of the mask. Mask 50 may optionally include deflector 38 and/or impermeable layer 39, which functions in the same fashion as described for mask 40 herein. Thus, as the wearer exhales breath, any portion of the exhaled breath that travels up the inner surface toward the eyes is blocked by one or more of bridge region 22 and leaflets 20 of partial inner seal 16, deflector 38 and/or impermeable layer 39. When the exhaled air is force back down, it can escape mask 50 from either or both of the sides or bottom of mask 50

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A face mask for filtering contaminants from air, comprising:
an air-permeable body having an inner surface, wherein said body is sized to cover the nose and mouth of a subject when worn by said subject;
an inner seal connected to said inner surface of said body, wherein said inner seal is sized to form a perimeter around a wearer's nostrils and mouth;
at least one strap connected to said body for securing said face mask to said subject's face; and
a deflector layer connected to the inner surface of said body, wherein the deflector redirects at least a portion of the exhaled air down into the lower regions of the air-permeable body.

2. The face mask of claim 1, wherein said inner seal is configured to form a seal around said subject's nose and mouth when worn by said subject.

3. The face mask of claim 1, wherein said inner seal comprises at least one leaflet for conforming a portion of said inner seal to said subject's nose.

4. The face mask of claim 1, wherein said inner seal comprises silicone.

5. The face mask of claim 1, wherein the at least one strap is connected to two or more extensions protruding from the side of the face mask.

6. The face mask of claim 1, wherein the air-permeable body is slightly rigid.

7. The face mask of claim 6, wherein an exterior surface of the air-permeable body includes at least one ridge to provide the slight rigidity.

8. The face mask of claim 1, wherein the deflector layer is air-impermeable.

9. The face mask of claim 8, wherein the deflector is at least partially coated with silicone along the inner surface of the face mask body.

10. A face mask for filtering contaminants from air, comprising:
an air permeable body having an inner surface, wherein said body is sized to cover the nose and mouth of a subject when worn by said subject;
an inner seal connected to said inner surface of said body, wherein said inner seal is sized to form a perimeter around a wearer's nostrils and mouth;
at least one strap connected to said body for securing said face mask to said subject's face; and
an air impermeable layer formed along the top portion of the inner surface of said body, wherein the air impermeable layer redirects at least a portion of the exhaled air down into the lower regions of the air-permeable body.

11. The face mask of claim 10, wherein said inner seal is configured to form a seal around said subject's nose and mouth when worn by said subject.

12. The face mask of claim 10, wherein said inner seal comprises at least one leaflet for conforming a portion of said inner seal to said subject's nose.

13. The face mask of claim 10, wherein said inner seal comprises silicone.

14. The face mask of claim 10, wherein the at least one strap is connected to two or more extensions protruding from the side of the face mask.

15. The face mask of claim 10, wherein the body is slightly rigid.

16. A face mask for filtering contaminants from air, comprising:
an air-permeable body having an inner surface, wherein said body is sized to cover the nose and mouth of a subject when worn by said subject;
an inner seal connected to said inner surface of said body, wherein said inner seal is sized to conform to the sides of the wearer's nose and cheek regions under the wearer's eyes;
at least one strap connected to said body for securing said face mask to said subject's face; and
a deflector layer connected to the inner surface of said body, wherein the deflector redirects at least a portion of the exhaled air down into the lower regions of the air-permeable body.

17. The face mask of claim 16, wherein said inner seal comprises at least one leaflet for conforming a portion of said inner seal to said subject's nose.

18. The face mask of claim 16, wherein said inner seal comprises silicone.

19. The face mask of claim 16, wherein the deflector layer is an air-impermeable layer.

20. The face mask of claim 19, wherein the deflector layer comprises a silicone coating.
